(19) United States
(12) Patent Application Publication
(10) Pub. No.: US 2023/0279462 A1
(43) Pub. Date: Sep. 7, 2023

(54) METHODS FOR DETECTING MICROORGANISMS

(71) Applicant: Spectacular Labs, Inc., Richmond, CA (US)

(72) Inventors: Okhtay Montazeri, Richmond, CA (US); Yuan-Sheng Fang, Oakland, CA (US)

(73) Assignee: Spectacular Labs, Inc., Richmond, CA (US)

(21) Appl. No.: 17/684,403

(22) Filed: Mar. 1, 2022

(51) Int. Cl. C12Q 1/04; C12Q 1/6888

(52) U.S. Cl. CPC C12Q 1/04; C12Q 1/6888

(57) ABSTRACT

A method of detection of a microorganism includes selecting test parameters and preparing a sample based on at least one of the test parameters. The method includes conducting tests on the sample based on the test parameters and providing test results and related data. The test results indicate the presence or absence of the microorganism. The method includes analyzing the test results and related data, and providing qualitative and quantitative assessments of the test results and related data. The method includes adjusting the test parameters to optimize the test parameters. The method includes repeating the tests with the adjusted test parameters, and adjusting the test parameters based on analyses of the test results and related data to optimize the test parameters.

### TECHNICAL FIELD

This description relates generally to methods for detecting microorganisms such as bacteria, viruses, or fungi.

### BACKGROUND

Microorganisms are abundant in the environment, and in fact, a large portion of the cells that exist in larger animals, such as humans are “external” microorganisms which do not share the same genetic material as the “host” species. Many of the microorganisms which exist in the environment or even within another “host” are either benign or even beneficial. Identifying the good bacteria, such as probiotics, and healthy gut flora may be useful for the overall health of the “host”, and that of the environment, as the growth and abundance of “good” microorganisms can hinder or suppress the growth of “bad” or pathogenic microorganisms. However, in certain circumstances, there may be special interest to detect the presence of pathogenic strains in food, or surfaces for certain applications.

Pathogens are infectious microorganisms such as bacteria, viruses, or fungi. In food and in the environment, the presence of pathogens can create significant health hazards. For example, food contaminated with Listeria, Salmonella or E. coli pose a serious threat to human health. Other pathogens such as tuberculosis pose a serious threat to livestock animals. Every single year, almost one sixth of the people in North America become ill as a result of exposure to foodborne pathogens.

Current methods for detecting the presence of microorganisms in food and in the environment have limitations which contribute to loopholes that allow microorganisms to go undetected. In most cases, current methods are not automated and at the same time easy-to-use, thus requiring trained operators to perform one or more steps. Also, in most cases, current methods do not accurately detect the presence of viable organisms, especially if a sample contains a low number of microorganisms. Furthermore, current methods are expensive, often requiring specialized laboratory environments for their operations in turn requiring the samples to be transported to these third-party laboratories, and as a result, there is a large lag time between sampling and results.

The background description provided here is for the purpose of generally presenting the context of the disclosure. Unless otherwise indicated here, the material described in this section is not prior art to the claims in this application and are not admitted being prior art by inclusion in this section.

### SUMMARY

In one aspect, a method of detection of a microorganism includes selecting test parameters and preparing a sample based on at least one of the test parameters. The method includes conducting tests on the sample based on the test parameters and providing test results and related data, wherein the test results indicate a presence or an absence of the microorganism. The method includes analyzing the test results and the related data and providing qualitative and quantitative assessments of the test results and the related data. The method includes adjusting the test parameters to optimize the test parameters. The method includes repeating the tests with the adjusted test parameters and adjusting the test parameters based on analyses of the test results and the related data to optimize the test parameters.

In an additional aspect, optimizing the test parameters includes increasing reliability of the detection of presence or absence of the microorganism, reducing an average time to obtain the test results, and/or reducing a cost of reagents used to perform the tests.

In an additional aspect, the method includes enriching the sample by diluting the sample with a liquid enrichment medium and incubating the diluted sample to allow levels of the microorganism to increase. The method includes lysing the enriched sample with a lysis solution to break down cells of the microorganism and to release nucleic compounds of the microorganism. The method includes amplifying the nucleic compounds to increase a number, or the length of a chain, of the nucleic compounds. The method includes detecting the presence or the absence of the microorganism by assaying the sample.

In an additional aspect, a machine-readable storage media includes machine-readable instructions stored thereon, that when executed, cause one or more machines to perform a method for detecting a microorganism in a sample. The method includes selecting test parameters and preparing a sample based on at least one of the test parameters and conducting tests on the sample based on the test parameters. The method includes providing test results and related data, wherein the test results indicate a presence or an absence of the microorganism. The method includes analyzing the test results and the related data and providing qualitative and quantitative assessments of the test results and the related data. The method includes adjusting the test parameters to optimize the test parameters. The method includes repeating the tests with the adjusted test parameters and adjusting the test parameters based on analyses of the test results and the related data to optimize the test parameters.

In an additional aspect, a system for detection of a microorganism includes a first module having interfaces configured to receive a sample and test parameters. The first module is operable to conduct tests on the sample and provide test results and related data. The test results indicate a presence or an absence of the microorganism in the sample. The system includes a second module having an interface configured to receive the test results and related data. The second module is operable to analyze the test results and related data and provide qualitative and quantitative assessments of the test results and related data. The system includes a third module having an interface configured to receive the qualitative and quantitative assessments and in response adjust the test parameters to optimize the test parameters. The adjusted test parameters are provided to the first module for subsequent tests.

In an additional aspect, a method of detection of a microorganism includes selecting test parameters and enriching a sample with least one of the test parameters. The method includes conducting tests on the enriched sample based on the test parameters and providing test results and related data, wherein the test results indicate a presence or an absence of the microorganism. The method includes analyzing the test results and the related data and providing qualitative and quantitative assessments of the test results and the related data. The method includes adjusting the test parameters to optimize the test parameters. The method includes repeating the tests with the adjusted test parameters and adjusting the test parameters based on analyses of the test results and the related data to optimize the test parameters.

### BRIEF DESCRIPTION OF THE DRAWINGS

The embodiments of the disclosure will be understood more fully from the detailed description given below and from the accompanying drawings of various embodiments of the disclosure, which, however, should not be taken to limit the disclosure to the specific embodiments, but are for explanation and understanding only.

FIG. 1 and FIG. 2 illustrate block diagrams of systems for detection of microorganisms in accordance with example embodiments.

FIG. 3 illustrates a flow diagram of a method of detection in accordance with an example embodiment.

FIG. 4 illustrates a flow diagram of a method of conducting tests in accordance with an example embodiment.

FIG. 5 illustrates a processor system in accordance with an example embodiment.

The same reference numerals or other reference designators are used in the drawings to designate the same or similar (by function and/or structure) features.

### DETAILED DESCRIPTION

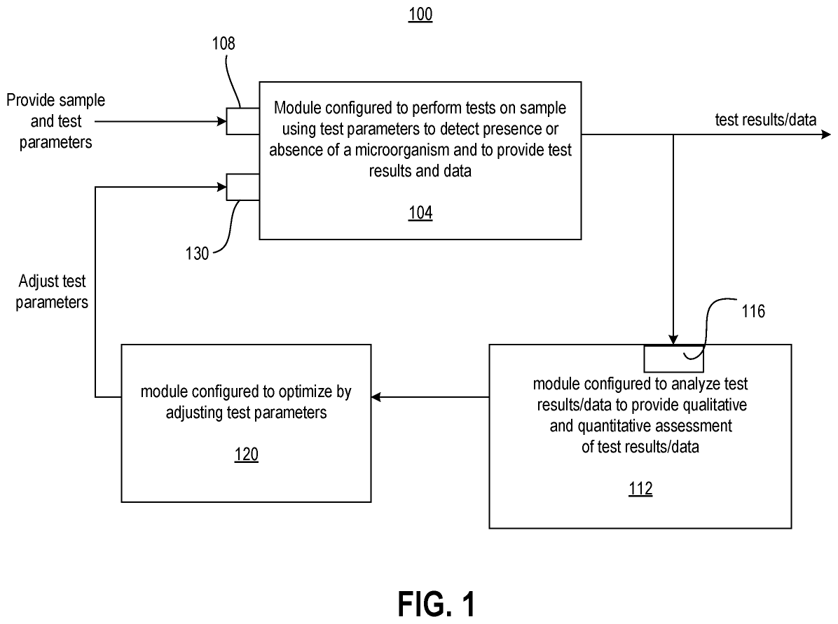

FIG. 1 illustrates a block diagram of a system 100 for detection of microorganisms in accordance with an example embodiment. The microorganisms may be any kind of bacterial, viral, microbial, fungal, or parasitic pathogens that are capable of multiplication.

In some embodiments, system 100 includes a module or platform 104 which includes one or more inputs or interfaces 108 configured to receive a sample and test parameters. The sample may be solid, semi-solid or liquid which may be contaminated with a microorganism. Any sampling method is encompassed, provided that it is suitable to acquire a sample of the microorganism of interest. A sample may be acquired by, for example, excision, blotting, swabbing, or sponging. Samples may include, but are not limited to meat, poultry, fish, produce, juices, dairy products, dry goods, raw and processed foods, tissue, urine, fecal matter, water, wastewater, soil, or surface samples. In some embodiments, the system 100 may include a plurality of modules or platforms 104 that are located at different physical locations.

The test parameters include, but are not limited to: reagents, supplements, diluents and other chemicals which are added to the sample; concentration and ratios of the reagents, supplements, diluents and other chemicals; temperature range; durations (e.g., incubation period, enrichment period).

In some example embodiments, interfaces 108 may be input ports configured to receive one or more cartridges or containers which may hold the sample, the reagents, the supplements, the diluents and other chemicals which are used for the tests. In some embodiments, module or platform 104 performs a series of tests to detect the presence or absence of the microorganism in the sample. The tests may be performed in an order; however, the order of the tests may be changed depending on the sample, or other criteria. Based on the tests, in some embodiments, module or platform 104 provides test results and related data which indicate the presence or absence of the microorganism of interest in the sample. For example, the test results may indicate the presence of the microorganism by a presumptive positive result and may indicate the absence of the microorganism by a presumptive negative result. The test results may also include the test parameters such as, but are not limited to: reagents, supplements, diluents and other chemicals which were added to the sample; such as, concentration and ratios of the reagents, supplements, diluents and other chemicals; raw data collected during the test; temperature range; durations (e.g., incubation period, enrichment period, total time for completion of the detection).

In some embodiments, system 100 includes a module or platform 112 having inputs or interfaces 116 configured to receive the test results and related data. In some example embodiments, module or platform 112 analyzes the test results and related data and provides qualitative and/or quantitative assessments of the test results and related data. As more tests are performed by module or platform 104, in some embodiments, module 112 repeats analysis of the test results and related data and provides qualitative and/or quantitative assessments.

In some example embodiments, a qualitative assessment of the test results and related data includes the detection of the presence or absence of the microorganism in the sample as well as the reliability or accuracy of the detection. The quantitative assessment of the test results and related data may include an assessment of the test parameters used in the test.

In some embodiments, system 100 includes a module or platform 120 which has an input or interface configured to receive the qualitative and/or quantitative assessments of the test results and related data. In response to the qualitative and/or quantitative assessments, module or platform 120 adjusts or updates one or more test parameters in order to optimize or modify the test parameters and improve performance of system 100.

In some embodiments, module or platform 104 includes an input or interface 130 configured to receive the adjusted or updated test parameters. In some embodiments, module or platform 104 performs subsequent tests using the adjusted or updated test parameters and provides test results and related data which are again analyzed by module or platform 112 to provide qualitative and/or quantitative assessments. In response, in some embodiments, module or platform 120 adjusts the test parameters to further optimize or modify the test parameters and improve performance of the system 100. In some example embodiments, the foregoing process is repeated for each test, and based on the qualitative and/or quantitative assessments, the test parameters are adjusted to optimize or modify the test parameters and improve the performance of system 100.

For example, module or platform 120 may vary the concentration and ratios of the reagents, supplements, diluents, and other chemicals and may vary the temperature and durations (e.g., incubation period, enrichment period) for optimization of the test parameters to improve performance of the system 100. In some embodiments, module or platform 120 may adjust the test parameters to increase reliability of the detection of presence or absence of the microorganism. Other parameters may be varied for optimization. For example, module 120 may adjust the test parameters to reduce the average time required to obtain test results and/or reduce the amount of reagents, supplements, and other chemicals to reduce the cost.

In some example embodiments, modules 112 and 120 include algorithm for optimizing various test parameters, criteria, or benchmarks. For example, the criteria or benchmarks can include, but are not limited to the average time to obtain test results, the average cost of reagents, supplements and other chemicals used to perform each test, or sensitivity of the detection of the microorganism of interest. In some embodiments, the algorithm may recommend if simultaneous tests for different nucleic sequences should be run as opposed to individual tests. In some embodiments, the algorithm may also recommend if tests should be performed at the onset of a specific event. In some embodiments, the algorithm may also recommend if the test sample should be preserved or transmitted to an outside laboratory for further testing. In some embodiments, the algorithm may issue a certification of the analysis if required conditions are met.

In some embodiments, module or platform 104 includes an input 130 for receiving the adjusted or updated test parameters. In some embodiments, module or platform 104 performs subsequent tests using the adjusted or updated test parameters and provides test results and related data.

In some example embodiments, module 104 is configured to detect if the sample contains a specific nucleic sequence. For example, milk from a dairy producer may be tested for the presence of Campylobacter or a sample of ground meat may be tested for the presence of E. coli bacteria.

In some example embodiments, modules 112 and 120 can be combined into a single module which can be configured to perform the functionalities of both modules 112 and 120.

In some example embodiments, modules or platforms 112 and 120 may be placed at various locations. In some embodiments, modules or platforms 112 and 120 may be a secure host service that can be hosted on an external server or on a cloud server. In some embodiments, module 104 may transmit test results and related data to an external server or a cloud server. In some embodiments, the external server or a cloud server may analyze the test results and related data and provide quantitative and/or qualitative assessments of the test results and related data and adjust or vary the test parameters to optimize or modify the test parameters and improve performance of system 100. In some embodiments, there may be multiple external servers or cloud servers that analyze the test results and related data. For example, different external servers or cloud servers may each analyze only parts of the test results and related data and based on their respective analysis provide qualitative and/or quantitative assessments which may be shared among the multiple external servers or cloud servers. The qualitative and/or quantitative assessments may be transmitted back to the module 104. In some embodiments, the system includes multiple modules 104 (i.e., multiple test modules 104) that are located at different physical locations. The different external servers or cloud servers may transmit their respective qualitative and/or quantitative assessments and adjusted test parameters back the multiple test modules 104. In response, the multiple test modules may conduct subsequent tests using the adjusted test parameters. In some embodiments, only a subset of the multiple test modules 104 may transmit the test results and related data to the external servers or the cloud servers which analyze the test results and related data and provide qualitative and quantitative assessments.

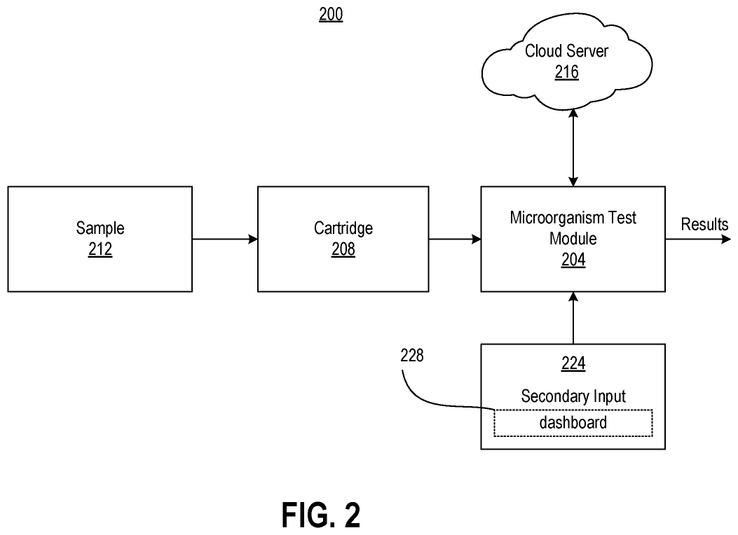

FIG. 2 is a block diagram of a system 200 of an example embodiment. In some embodiments, system 200 is an example implementation of system 100 illustrated in FIG. 1. In some embodiments, system 200 includes a module or platform 204 (microorganism test module 204) configured to run tests to detect the presence or absence of microorganism in a test sample. In some embodiments, module or platform 204 may be in the form of a box that has one or more receptacles or bins for receiving one or more cartridges or containers 208. In some embodiments, cartridges 208 may be pre-loaded with reagents, supplements, diluents and other materials. In some embodiments, a sample 212, which may be contaminated with the microorganism, can be loaded into cartridge 208, and cartridge 208 is then be loaded into module or platform 204.

In some embodiments, system 200 includes a module 216 (e.g., cloud server 216, external server, local computing machine, mobile device, etc.) which may be placed at various locations. In some embodiments, module 216 may be a secure host service that can be hosted on an external server or on a cloud server. In some embodiments, platform 204 may transmit the test results and related data to module 216. In some example embodiments, module 216 is an external server or a cloud server. In some embodiments, external server or a cloud server may include algorithm for analyzing the test results and related data. In some embodiments, the algorithm analyzes the test results and related data, and based on the analysis provides quantitative and/or qualitative assessments of the test results and related data. Based on the qualitative and/or quantitative assessments, in some embodiments the algorithm adjusts or varies the test parameters to optimize or modify the test parameters and improve performance of system 200. In some embodiments, the qualitative and/or quantitative assessments and the adjusted or updated test parameters are transmitted back to module or platform 204. In some embodiments, the adjusted or updated test parameters are used by module or platform 204 to run subsequent tests. As such, the test results and related data are used by system 200 to optimize or modify subsequent test parameters and improve performance of system 200.

In some embodiments, system 200 includes a user interface 224 configured to enable a user to configure module or platform 204. A user may provide secondary inputs to module platform 204. For example, a user may set various test parameters (e.g., temperature, duration, ratios) via user interface 224. In some embodiments, user interface 224 may include a dashboard 228 configured to interact with a user. In some embodiments, dashboard 228 may be configured to receive results of the analysis and visualize test results. In some embodiments, dashboard 228 may be configured to track test status, results, and test schedules.

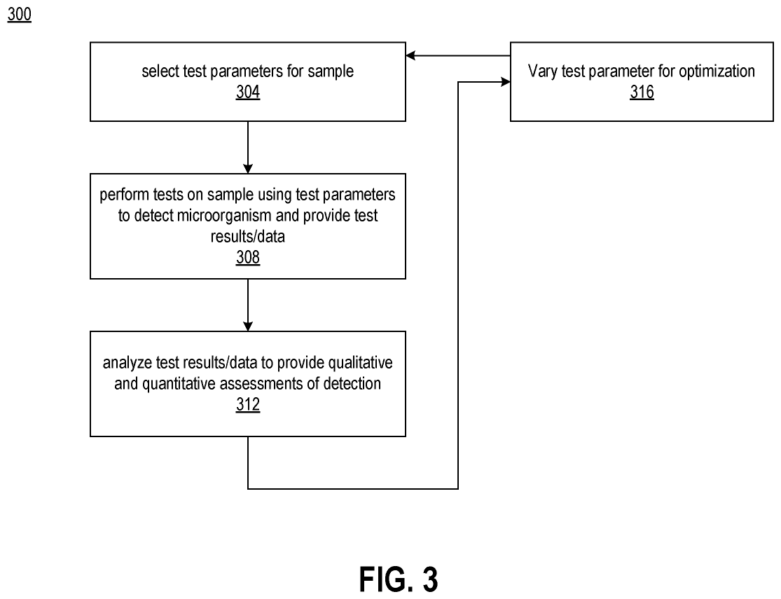

FIG. 3 illustrates a flow diagram of a method 300 of detection in accordance with an example embodiment. In block 304, test parameters are selected for a sample. The order of various blocks shown here can be modified. For example, some blocks may be performed simultaneously. In some embodiments, the various blocks shown here can be implemented in hardware, software, or a combination of them. The sample (e.g., sample 212) may be solid, semi-solid or liquid which may be contaminated with a microorganism. The test parameters include, but are not limited to: reagents, supplements, diluents and other chemicals which are added to the sample; concentration and ratios of the reagents, supplements, diluents and other chemicals; temperature range; durations (e.g., incubation period, enrichment period). These parameters can be pre-loaded into cartridge 208, in accordance with some embodiments.

In block 308, tests are performed on the sample using the test parameters and test results and related data are provided. For example, test module 204 executes a process discussed herein to detect the microorganism of interest. The test results indicate the presence or absence of the microorganism in the sample. For example, the test results may indicate the presence of the microorganism by a presumptive positive result and may indicate the absence of the microorganism by a presumptive negative result. The test results and related data may include the test parameters such as, but not limited to: reagents, supplements, diluents and other chemicals which were added to the sample; concentration and ratios of the reagents, supplements, diluents and other chemicals; raw data collected during the test; temperature range; durations (e.g., incubation period, enrichment period, total time for completion of the detection).

In block 312, test results and related data are analyzed and qualitative and/or quantitative assessments of the test results and related data are provided. In one example, the test results are analyzed by cloud server 216. In other examples, local or remote computing devices may be used to analyze the data. The qualitative assessment of the test results and related data may include the reliability or accuracy of the detection. The quantitative assessment of the test results and related data may include the time to obtain test results.

In block 316, responsive to the qualitative and/or quantitative assessments of the test results and related data, the test parameters are adjusted to optimize or modify the test parameters and improve performance of the system. For example, module 120 or cloud server 216 may optimize the test parameters in response to the qualitative and/or quantitative assessments. The flow returns to block 304 where subsequent tests are performed using the adjusted or updated test parameters. In some example embodiments, the foregoing process is repeated for each test, and based on the qualitative and/or quantitative assessments, the test parameters are adjusted to optimize or modify the test parameters and improve the performance of the system.

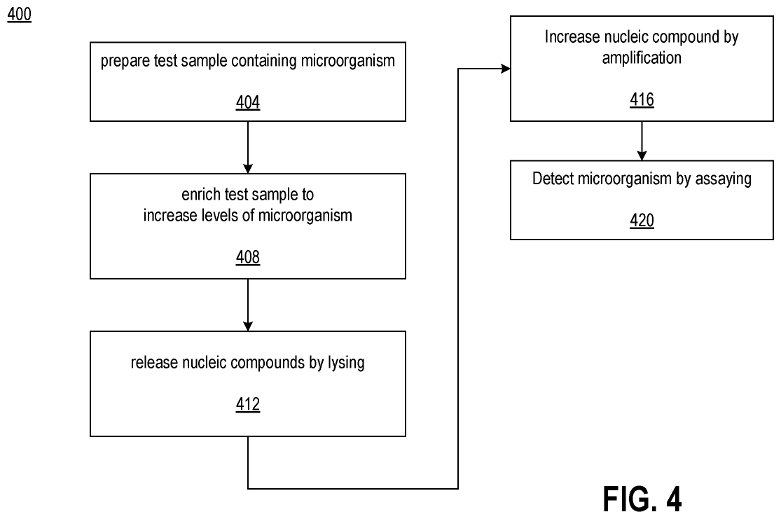

FIG. 4 illustrates a flow diagram of a method 400 of conducting tests in accordance with an example embodiment. The order of various blocks shown here can be modified. For example, some blocks may be performed simultaneously. In some embodiments, the various blocks shown here can be implemented in hardware, software, or a combination of them. In block 404, a test sample (e.g., sample 212) is prepared. The sample may be contaminated with the microorganism. In block 408, the sample is enriched by diluting the sample with a liquid enrichment medium at a first ratio of the sample to the diluent and incubating the diluted sample for a first time period to allow levels of the microorganism to increase.

In block 412, the enriched sample is lysed with a lysis solution at a second ratio of the enriched solution to the lysis solution to break down cells of the microorganism and to release nucleic compounds of the microorganism. For example, the enriched sample may be lysed in microorganism test module 204. In block 416, the nucleic compounds are amplified to increase the number of nucleic compounds. For example, the nucleic compounds may be amplified in microorganism test module 204. In block 420, the sample is assayed to detect the presence or absence of the microorganism. For example, the sample may be assayed in microorganism test module 204.

By way of example, a test sample is made using the following method. A first mixture containing a sample and suitable amount of phosphate-buffered water is blended. A second mixture is made by adding Bolton antibiotic additive and lysed horse blood to a Bolton broth. A third mixture is made by adding the first mixture and the second mixture, and the pH of the third mixture is adjusted to approximately 7.5. The third mixture is rotated in a centrifuge to separate the test sample from the remaining mixture. The test sample is separated as pellet.

As an example, the test sample is enriched by incubating the sample at 37 degrees C. for a period of 4 hours or at 42 degrees C. for a period of 48 hours, and the test sample is pre-amplified by adding enzymes, reactants, or primers. In some example embodiments, the presence of the microorganism is detected by the presence of a biomarker or bioluminescence.

In some example embodiments, a computer program product comprising a computer readable medium includes computer program logic for the detection of a microorganism in a sample. The computer program logic includes program code to select test parameters for the sample; program code to conduct tests on the sample based on the test parameters and provide test results and related data, wherein the test results indicate the presence or absence of the microorganism; program code to analyze the test results and related data and provide qualitative and quantitative assessments of the test results and related data; program code to adjust the test parameters to optimize or modify the test results; and program code to repeat the tests with the adjusted test parameters and to adjust the test parameters based on analyses of the test results and related data to optimize the test results.

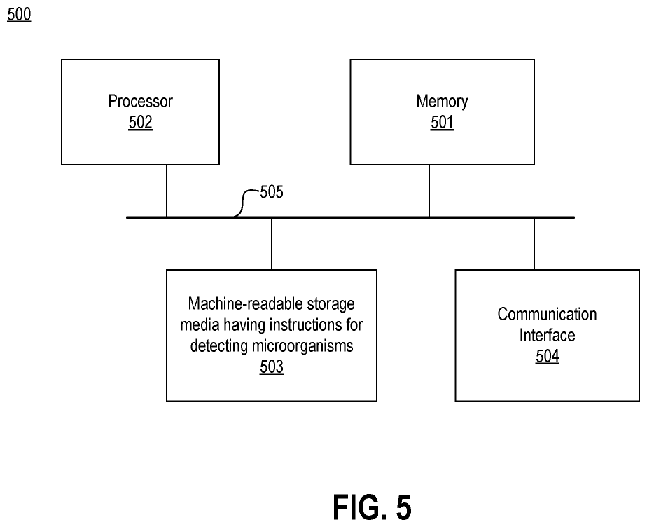

FIG. 5 illustrates a processor system 500 with machine-readable storage media having instructions that when executed cause a machine to detect the presence or absence of a microorganism and to optimize test parameters. Processes described in various embodiments of the present disclosure may be stored in a machine-readable medium as computer-executable instructions. In some embodiments, processor system 500 comprises memory 501, processor 502, machine-readable storage media 503 (also referred to as tangible machine-readable medium), communication interface 504 (e.g., wireless or wired interface), and network bus 505 coupled together as shown. In some embodiments, the various components of system 500 may be part of processor 502.

In some embodiments, processor 502 is a Digital Signal Processor (DSP), an Application Specific Integrated Circuit (ASIC), a general-purpose Central Processing Unit (CPU), or a low power logic implementing a simple finite state machine to perform various processes described herein.

In some embodiments, the various logic blocks of processor system 500 are coupled together via network bus 505. Any suitable protocol may be used to implement network bus 505. In some embodiments, machine-readable storage medium 503 includes instructions (also referred to as the program software code/instructions) for the detection of microorganism in a sample, as described above with reference to various embodiments.

In one example, machine-readable storage media 503 is a machine-readable storage media with instructions for the detection of microorganism in a sample (herein machine-readable medium 503) and for providing test results and related data. Machine-readable medium 503 has machine-readable instructions, that when executed, cause processor 502 to perform the method as discussed with reference to various embodiments.

Program software code/instructions associated with various embodiments may be implemented as part of an operating system or a specific application, component, program, object, module, routine, or other sequence of instructions or organization of sequences of instructions referred to as “program software code/instructions,” “operating system program software code/instructions,” “application program software code/instructions,” or simply “software” or firmware embedded in processor. In some embodiments, the program software code/instructions associated with processes of various embodiments are executed by processor system 500.

In some embodiments, machine-readable storage media 503 is a computer-executable storage medium 503. In some such embodiments, the program software code/instructions associated with various embodiments are stored in computer-executable storage medium 503 and executed by processor 502. Here, computer executable storage medium 503 is a tangible machine-readable medium 503 that can be used to store program software code/instructions and data that, when executed by a computing device, causes one or more processors (e.g., processor 502) to perform a process.

The tangible machine-readable medium 503 may include storage of the executable software program code/instructions and data in various tangible locations, including for example ROM, volatile RAM, non-volatile memory and/or cache and/or other tangible memory as referenced in the present application. Portions of this program software code/instructions and/or data may be stored in any one of these storage and memory devices. In some embodiments, the program software code/instructions can be obtained from other storage, including, e.g., through centralized servers or peer-to-peer networks and the like, including the Internet. Different portions of the software program code/instructions and data can be obtained at different times and in different communication sessions or in the same communication session.

The software program code/instructions associated with the various embodiments can be obtained in their entirety prior to the execution of a respective software program or application. Alternatively, portions of the software program code/instructions and data can be obtained dynamically, e.g., just in time, when needed for execution. Alternatively, some combination of these ways of obtaining the software program code/instructions and data may occur, e.g., for different applications, components, programs, objects, modules, routines or other sequences of instructions or organization of sequences of instructions, by way of example. Thus, it is not required that the data and instructions be on a tangible machine-readable medium 503 in entirety at a particular instance of time.

Examples of tangible machine-readable medium 503 include but are not limited to recordable and non-recordable type media such as volatile and non-volatile memory devices, read only memory (ROM), random-access-memory (RAM), flash memory devices, floppy and other removable disks, magnetic storage media, optical storage media (e.g., Compact Disk Read-Only Memory (CD ROMS), Digital Versatile Disks (DVDs), etc.), among others. The software program code/instructions may be temporarily stored in digital tangible communication links while implementing electrical, optical, acoustical, or other forms of propagating signals, such as carrier waves, infrared signals, digital signals, etc. through such tangible communication links.

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs or codes as described herein and elsewhere may be executed by the server. In addition, other devices required for execution of methods as described in this application may be considered as a part of the infrastructure associated with the server. The server may provide an interface to other devices including, without limitation, clients, other servers, printers, database servers, print servers, file servers, communication servers, distributed servers, social networks, and the like.

Additionally, communication via a wired link or a wireless link may facilitate remote execution of program across the network. The networking of some or all these devices may facilitate parallel processing of a program or method at one or more location without deviating from the scope of the invention. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client, and the like. The client may include one or more of memories, processors, computer readable media, storage media, ports (physical and virtual), communication devices, and interfaces capable of accessing other clients, servers, machines, and devices through a wired or a wireless medium, and the like. The methods, programs or codes as described herein and elsewhere may be executed by the client. In addition, other devices required for execution of methods as described in this application may be considered as a part of the infrastructure associated with the client.

The networking of some or all these devices may facilitate parallel processing of a program or method at one or more location without deviating from the scope of the invention. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, cloud servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM, and the like. The processes, methods, program codes, instructions described herein and elsewhere may be executed by one or more of the network elements.

Those skilled in the art will recognize that, for simplicity and clarity, the full structure and operation of all systems suitable for use with the present disclosure is not being depicted or described herein. Instead, only so much of a system as is unique to the present disclosure or necessary for an understanding of the present disclosure is depicted and described. The remainder of the construction and operation of the disclosed systems may conform to any of the various current implementations and practices known in the art.

Of course, those of skill in the art will recognize that, unless specifically indicated or required by the sequence of operations, certain steps in the processes described above may be omitted, performed concurrently or sequentially, or performed in a different order. Further, no component, element, or process should be considered essential to any specific claimed embodiment, and each of the components, elements, or processes can be combined in still other embodiments.

Modifications are possible in the described embodiments, and other embodiments are possible, within the scope of the claims.

Throughout the specification, and in the claims, the term “connected” means a direct connection, such as electrical, mechanical, or magnetic connection between the things that are connected, without any intermediary devices.

The term “coupled” means a direct or indirect connection, such as a direct electrical, mechanical, or magnetic connection between the things that are connected or an indirect connection, through one or more passive or active intermediary devices.

The term “adjacent” here generally refers to a position of a thing being next to (e.g., immediately next to or close to with one or more things between them) or adjoining another thing (e.g., abutting it).

The term “circuit” or “module” may refer to one or more passive and/or active components that are arranged to cooperate with one another to provide a desired function.

The terms “substantially,” “close,” “approximately,” “near,” and “about,” generally refer to being within +/−10% of a target value.

Unless otherwise specified the use of the ordinal adjectives “first,” “second,” and “third,” etc., to describe a common object, merely indicate that different instances of like objects are being referred to and are not intended to imply that the objects so described must be in a given sequence, either temporally, spatially, in ranking or in any other manner.

For the purposes of the present disclosure, phrases “A and/or B” and “A or B” mean (A), (B), or (A and B). For the purposes of the present disclosure, the phrase “A, B, and/or C” means (A), (B), (C), (A and B), (A and C), (B and C), or (A, B and C).

The terms “left,” “right,” “front,” “back,” “top,” “bottom,” “over,” “under,” and the like in the description and in the claims, if any, are used for descriptive purposes and not necessarily for describing permanent relative positions.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may,” “might,” or “could” be included, that particular component, feature, structure, or characteristic is not required to be included. If the specification or claim refers to “a” or “an” element, that does not mean there is only one of the elements. If the specification or claims refer to “an additional” element, that does not preclude there being more than one of the additional elements.

Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive.

While the disclosure has been described in conjunction with specific embodiments thereof, many alternatives, modifications and variations of such embodiments will be apparent to those of ordinary skill in the art considering the foregoing description. The embodiments of the disclosure are intended to embrace all such alternatives, modifications, and variations as to fall within the broad scope of the appended claims. Where specific details are set forth to describe example embodiments of the disclosure, it should be apparent to one skilled in the art that the disclosure can be practiced without, or with variation of, these specific details. The description is thus to be regarded as illustrative instead of limiting.

1. A method of detection of a microorganism, comprising:
selecting test parameters and preparing a sample based on at least one of the test parameters;
conducting tests on the sample based on the test parameters and providing test results and related data, wherein the test results indicate a presence or an absence of the microorganism;
analyzing the test results and the related data and providing qualitative and quantitative assessments of the test results and the related data;
adjusting the test parameters to optimize the test parameters; and
repeating the tests with the adjusted test parameters and adjusting the test parameters based on analyses of the test results and the related data to optimize the test parameters.

2. The method of claim 1, wherein optimizing the test parameters includes increasing reliability of the detection of presence or absence of the microorganism.

3. The method of claim 1, wherein optimizing the test parameters includes reducing an average time to obtain the test results.

4. The method of claim 1, wherein optimizing the test parameters includes reducing a cost of reagents used to perform the tests.

5. The method of claim 1, wherein conducting the tests comprises:
enriching the sample by diluting the sample with a liquid enrichment medium;
incubating the diluted sample to allow levels of the microorganism to increase;
lysing the enriched sample with a lysis solution to break down cells of the microorganism and to release nucleic compounds of the microorganism;
amplifying the nucleic compounds to increase a number of the nucleic compounds; and
detecting the presence or the absence of the microorganism by assaying the sample.

6. The method of claim 5, further comprising detecting the presence of the microorganism by detecting the presence of a biomarker in the sample.

7. The method of claim 5, further comprising detecting the presence of the microorganism by detecting bioluminescence in the sample.

8. A machine-readable storage media having machine-readable instructions stored thereon, that when executed, cause one or more machines to perform a method for detecting a microorganism in a sample, the method comprising:
selecting test parameters and preparing a sample based on at least one of the test parameters;
conducting tests on the sample based on the test parameters and providing test results and related data, wherein the test results indicate a presence or an absence of the microorganism;
analyzing the test results and the related data and providing qualitative and quantitative assessments of the test results and the related data;
adjusting the test parameters to optimize the test parameters; and
repeating the tests with the adjusted test parameters and adjusting the test parameters based on analyses of the test results and the related data to optimize the test parameters.

9. The machine-readable storage media of claim 8, wherein optimizing the test parameters includes increasing reliability of the detection of presence or absence of the microorganism.

10. The machine-readable storage media of claim 8, wherein optimizing the test parameters includes reducing an average time to obtain the test results.

11. The machine-readable storage media of claim 8, wherein optimizing the test parameters includes reducing a cost of reagents used to perform the tests.

12. The machine-readable storage media of claim 8, wherein conducting the tests comprises:
enriching the sample by diluting the sample with a liquid enrichment;
incubating the diluted sample to allow levels of the microorganism to increase;
lysing the enriched sample with a lysis solution to break down cells of the microorganism and to release nucleic compounds of the microorganism;
amplifying the nucleic compounds to increase a number of the nucleic compounds; and
detecting the presence or the absence of the microorganism by assaying the sample.

13. The machine-readable storage media of claim 8, having further machine-readable instructions stored thereon, that when executed, cause the one or more machines to perform a further method for detecting a microorganism in a sample, comprising:
detecting the presence of the microorganism by detecting the presence of a biomarker in the sample.

14. The machine-readable storage media of claim 8, having further machine-readable instructions stored thereon, that when executed, cause the one or more machines to perform a further method for detecting a microorganism in a sample, comprising:
detecting the presence of the microorganism by detecting bioluminescence in the sample.

15. A system for detection of a microorganism, comprising:
a first module having interfaces configured to receive a sample and test parameters, the first module operable to conduct tests on the sample and provide test results and related data, wherein the test results indicate a presence or an absence of the microorganism in the sample;
a second module having an interface configured to receive the test results and related data, the second module operable to analyze the test results and related data and provide qualitative and quantitative assessments of the test results and related data; and
a third module having an interface configured to receive the qualitative and quantitative assessments and in response adjust the test parameters to optimize the test parameters,
wherein the adjusted test parameters are provided to the first module for subsequent tests.

16. The system of claim 15, wherein the first module is configured to enrich the sample by diluting the sample with a liquid enrichment medium and incubating the diluted sample.

17. The system of claim 16, wherein the first module is configured to lyse the enriched sample with a lysis solution to break down cells of the microorganism and to release nucleic compounds of the microorganism.

18. The system of claim 17, wherein the first module is configured to amplify the nucleic compounds to increase a number of the nucleic compounds.

19. The system of claim 17, wherein the first module is configured to amplify the nucleic compounds to increase a length of a chain of the nucleic compounds.

20. The system of claim 18, wherein the first module is configured to detect the presence or the absence of the microorganism by assaying the sample.

21. The system of claim 15, wherein:
the test parameters are modified to increase a reliability of the detection of the presence or the absence of the microorganism;
the test parameters are modified to reduce an average time to obtain test results; or
the test parameters are modified to reduce a cost of reagents used to perform the tests.

22. A method of detection of a microorganism, comprising:
selecting test parameters;
enriching a sample with least one of the test parameters;
conducting tests on the enriched sample based on the test parameters and providing test results and related data, wherein the test results indicate a presence or an absence of the microorganism;
analyzing the test results and the related data and providing qualitative and quantitative assessments of the test results and the related data;
adjusting the test parameters to optimize the test parameters; and
repeating the tests with the adjusted test parameters and adjusting the test parameters based on analyses of the test results and the related data to optimize the test parameters.
